(19) United States
(12) Patent Application Publication
(10) Pub. No.: US 2015/0374935 A1
(43) Pub. Date: Dec. 31, 2015

(54) GASIFICATION APPARATUS, GASIFICATION ASSEMBLY KIT AND METHOD FOR GASIFICATION AND CONCENTRATION OF A GASIFIABLE COMPOUND

(71) Applicant: Enrico BOUCHARD, Aldergrove (CA)

(72) Inventor: Enrico BOUCHARD, Aldergrove (CA)

(21) Appl. No.: 14/767,176

(22) PCT Filed: Feb. 11, 2014
(86) PCT No.: PCT/CA2014/050090

§ 371 Date: Aug. 11, 2015

Related U.S. Application Data
(60) Provisional application No. 61/763,248, filed on Feb. 11, 2013.

(51) Int. Cl. A61M 11/04; A61M 15/06; A61M 11/06

(52) U.S. Cl. CPC A61M 11/042; A61M 11/06; A61M 15/06

(57) ABSTRACT

A gasification apparatus gasifies and concentrates a gasifiable compound. The gasification apparatus includes a closure member having a closure member body and a gasification body engageable with the closure member to define a gasification chamber. The gasification chamber is in gas communication with at least one air intake defined in at least one of the gasification body and the closure member. The gasification body is in heat conductive transfer with the closure member. The gasification body includes a gasifiable source material support extending below the gasification chamber and at least one concentration channel in gas communication with the gasification chamber. A heating member operatively connects to at least one of the closure member body and the gasification body for heating the same. An assembly kit and method of operation are for gasification and concentration.

### CROSS REFERENCE TO RELATED APPLICATIONS

This application claims priority under 35 USC §119(e) of United-States provisional patent application No. 61/763,248 filed on Feb. 11, 2013, which is hereby incorporated by reference.

### FIELD OF THE INVENTION

The present invention relates to the field of gasification of gasifiable compounds. More particularly, it relates to an apparatus, an assembly kit and a method for the gasification and concentration of a gasifiable compound found in a source material such as herbs or other blends, before the gasifiable compound is inhaled by a user.

### BACKGROUND

It is well known that the combustion of source materials (e.g. plant materials or other herbs or blends), in order to extract the active ingredients therefrom, often generates irritating, toxic and/or carcinogenic source materials that are by-products of the combustion. Consequently, subsequent inhalation of the smoke created by the combustion process of the source material can be a major health hazard for users, and especially for frequent users.

However, extraction of the active ingredient from a source material, to be subsequently inhaled by a user, is desirable in many cases, for example for individuals having a condition justifying the inhalation of such active ingredients from the source material in order to attenuate diverse symptoms.

Therefore, different alternatives have been proposed to allow users to extract volatile compounds or active ingredients from a source material, while minimizing the quantity of by-products inhaled by the users. Such alternatives comprise devices commonly known as “vaporizers”, which are found on the market. These devices are designed to heat the source material in order to perform the gasification of volatile compounds found in the source material without combustion of the source material. When the source material is gasified rather than burned, substantially only the compounds or active ingredients which are sufficiently volatile to evaporate at a given temperature will be gasified and will subsequently be inhaled by the user.

Known vaporizers usually perform gasification of the volatile compound of a source material by heating the source material either by thermal conduction, by convection, or by thermal radiation. For thermal conduction, the source material is usually heated by a heated metal plate onto which the source material is placed. For thermal convection, the source material is heated by hot air circulating through it, thereby causing gasification of the volatile compound found in the source material. Finally, for thermal radiation, the source material is heated by radiant energy, e.g. by using a superheated thermal mass positioned proximate to the source material, in order to rise the temperature of the source material until the volatile compound found in the source material is gasified.

One of the drawbacks of the use of a single heating method to perform gasification of the volatile compound is the frequent occurrence of incomplete gasification of the volatile compound found in the source material. Moreover, using known vaporisers, once the volatile compound has been extracted from the source material by gasification, the extracted gas is usually delivered to the user as such. Consequently, a greater quantity of source material is required in order to treat the symptoms of a user.

In view of the above, there is a need for an improved apparatus, assembly kit and method, which would be able to overcome or at least minimize some of the above-discussed prior art concerns.

### SUMMARY OF THE INVENTION

According to a first general aspect, there is provided a gasification apparatus for gasifying and concentrating a gasifiable compound. The gasification apparatus comprises a closure member thermally couplable to a heating member configured to heat the closure member and a receiving member having a receiving section and a treatment gas outlet in gas communication with the receiving section. The closure member is engageable with the receiving member to close the receiving section and define a gasification chamber in between having at least one air intake. The receiving member is in thermal exchange with the closure member when they are engaged together. The gasification apparatus also comprises a concentration member in thermal communication with the receiving member and in gas communication with the treatment gas outlet of the receiving member. The concentration member has at least one concentration channel with an internal volume configured to cause a treatment gas flowing from the gasification chamber to concentrate into a concentrated treatment gas.

In an embodiment, the receiving member further comprises a gasifiable source material support mounted in the receiving section between the at least one air intake and the treatment gas outlet.

In an embodiment, the gasifiable source material support is gas permeable and is mounted upstream of the treatment gas outlet.

In an embodiment, the closure member comprises an injector having an injector body. The at least one air intake comprises at least one air inlet port defined in the injector body and being in gas communication with at least one air outlet port defined in the injector body. The at least one air outlet port is located in the receiving section when the closure member and the receiving member are engaged together.

In an embodiment, the at least one air inlet port comprises at least one aperture defined in a periphery of an inlet section of the injector and the at least one air outlet port comprises at least one aperture defined in a periphery of an outlet section of the injector.

In an embodiment, the at least one air inlet port comprises a plurality of air inlet ports and the at least one air outlet port comprises a plurality of air outlet ports, each one of the air inlet ports being in gas communication with a respective one of the air outlet ports.

In an embodiment, the injector further comprises a concentrated source material receptacle for receiving a concentrated source material, the concentrated source material receptacle being configured to allow at least a portion of a gas generated from the concentrated source material to be drawn into at least one of the at least one air inlet port.

In an embodiment, the concentrated source material receptacle comprises a cavity defined in an inlet wall of the injector and the at least one air inlet port comprises a plurality of air inlet ports, the cavity being surrounded by the air inlet ports in gas communication with the at least one air outlet port.

In an embodiment, the gasification apparatus further comprises a concentrated treatment gas output tube connectable to the concentration member and being in gas communication with the concentration member when connected thereto. The concentrated treatment gas output tube defines a concentrated treatment gas output channel having an inner diameter greater than an inner diameter of the at least one concentration channel of the concentration member.

In an embodiment, the gasification apparatus further comprises an inhalation component connectable to the concentrated treatment gas output tube and being in gas communication therewith when connected together. The inhalation component has an inhaling outlet port allowing inhalation of the concentrated treatment gas by a user.

In an embodiment, the internal volume of the at least one concentration channel formed in the concentration member is between about 20 mm³ and about 50 mm³.

In an embodiment, the internal volume of the at least one concentration channel formed in the concentration member is between about 20 mm³ and about 30 mm³.

According to another general aspect, there is also provided a gasification apparatus for gasifying and concentrating a gasifiable compound. The gasification apparatus comprises a closure member having a closure member body and a gasification body engageable with the closure member to define a gasification chamber in between. The gasification body is in heat conductive transfer with the closure member when engaged together. The gasification chamber is in gas communication with at least one air intake defined in at least one of the gasification body and the closure member body. The gasification body includes at least one concentration channel in gas communication with the gasification chamber. The gasification apparatus also comprises a heating member operatively connectable to at least one of the closure member body and the gasification body for heating same.

According to another general aspect, there is also provided a gasification assembly kit for an apparatus to gasify and concentrate a gasifiable compound. The gasification assembly kit comprises a closure member and a receiving member with a receiving section and a treatment gas outlet. The receiving section is sized and shaped to be connectable with the closure member to define a gasification chamber in gas communication with the treatment gas outlet. The gasification chamber is in gas communication with at least one air intake defined in at least one of the receiving section and the closure member. The gasification assembly kit also comprises a heating member thermally connectable to at least one of the closure member and the receiving member, and a gas concentration member engageable with the receiving member and having at least one concentration channel. The at least one concentration channel is in gas communication with the treatment gas outlet when the gas concentration member and the receiving member are engaged together.

According to another general aspect, there is further provided a method of gasifying and concentrating a gasifiable compound found in a source material. The method comprises the steps of: heating at least a closure member and a receiving member until a temperature set-point is reached and the closure member and the receiving member are in a substantial thermal equilibrium; inserting the source material into a gasification chamber defined between the closure member and the receiving member, when engaged together; generating a treatment gas from the gasifiable compound of the source material with the closure member and the receiving member being engaged together; and concentrating the treatment gas into a concentrated treatment gas by passing the treatment gas into at least one concentration channel of a concentration member in gas communication with the gasification chamber.

In an embodiment, the step of inserting the source material into the gasification chamber occurs prior to the heating of the closure member and the receiving member.

In an embodiment, the step of inserting the source material into the gasification chamber occurs concurrently with the heating of the closure member and the receiving member.

In an embodiment, the temperature set-point corresponds to a temperature wherein the gasifiable compound at least partially gasifies.

In an embodiment, the temperature set-point is between about 190° Celsius and about 240° Celsius.

In an embodiment, the step of heating at least the closure member and the receiving member until a temperature set-point is reached further comprises heating the concentration member until the closure member, the receiving member and the concentration member are in a substantial thermal equilibrium.

In an embodiment, the step of generating a treatment gas is performed by at least one of conduction heating, convection heating and radiation heating between the closure member, the receiving member, and the source material.

In an embodiment, the step of generating a treatment gas is performed by at least two of conduction heating, convection heating and radiation heating between the closure member, the receiving member, and the source material.

In an embodiment, the step of generating a treatment gas is performed by conduction heating, convection heating and radiation heating between the closure member, the receiving member, and the source material.

In an embodiment, the step of generating a treatment gas is performed by inhaling the treatment gas from a gas outlet port, the gas outlet port being in gas communication with the at least one concentration channel, the gasification chamber, and at least one air intake defined in one of the closure member and the receiving member.

According to another general aspect, there is provided an apparatus for gasifying and concentrating a gasifiable compound. The apparatus comprises an injector having at least one air inlet port and at least one air outlet port. The injector is thermally coupled to a heating member which heats the injector. The apparatus further comprises a receiving member having a receiving section for receiving a source material comprising the gasifiable compound, and a treatment gas outlet in gas communication with the receiving section. The injector is connectable to the receiving member so as to heat the receiving member and close the receiving section. The apparatus further comprises a concentration member in thermal communication with the receiving member and in gas communication with the treatment gas outlet. The concentration member defines at least one concentration channel with an internal volume causing a treatment gas generated in the receiving member to concentrate into a concentrated treatment gas.

According to another general aspect, there is also provided an apparatus for gasifying and concentrating a gasifiable compound. The apparatus comprises a closure member having a closure member body and a gasification body engageable with the closure member to define a gasification chamber. The gasification chamber is in gas communication with at least one air intake defined in at least one of the gasification body and the closure member and is in heat conductive transfer with the closure member. The gasification body includes a gasifiable source material support extending below the gasification chamber and at least one concentration channel in gas communication with the gasification chamber. The apparatus further comprises a heating member operatively connected to at least one of the closure member body and the gasification body for heating the same.

According to yet another general aspect, there is also provided an assembly kit for an apparatus to gasify and concentrate a gasifiable compound. The assembly kit comprises a closure member and a receiving member with a receiving section. The receiving section is sized and shaped to receive a source material comprising the gasifiable compound and to be connectable with the closure member to define a gasification chamber having a treatment gas outlet port. The gasification chamber is in gas communication with at least one air intake defined in at least one of the receiving section and the closure member. The assembly kit further comprises a heating member thermally connectable to at least one of the closure member and the receiving member and a gas concentration member connectable to the treatment gas outlet port such that the gas concentration member is in gas communication with the treatment gas outlet port of the gasification chamber.

According to yet another general aspect, there is also provided a method of gasifying and concentrating a gasifiable compound found in a source material. The method comprises the step of providing an injector, a receiving member and a concentration member, the injector and receiving member forming a gasification chamber when engaged to one another. The method further comprises the step of heating the injector and the receiving member until a temperature set-point is reached and the injector and the receiving member are in a substantial thermal equilibrium. The temperature set-point corresponds to a temperature wherein the gasifiable compound at least partially gasifies. The method further comprises the steps of inserting the source material into the gasification chamber, generating a treatment gas from the gasifiable compound of the source material, and concentrating the treatment gas into a concentrated treatment gas by passing the treatment gas into at least one concentration channel of the concentration member.

### BRIEF DESCRIPTION OF THE DRAWINGS

Other objects, advantages and features will become more apparent upon reading the following non-restrictive description of embodiments thereof, given for the purpose of exemplification only, with reference to the accompanying drawings in which:

FIG. 1 is an exploded view of components of a gasification apparatus, in accordance with an embodiment where a concentration member is connectable with a receiving member.

FIG. 2 is an exploded view of components of a gasification apparatus, in accordance with another embodiment where the concentration member is integral with the receiving member.

FIG. 3 is an exploded view of an injector and an associated heating member of the apparatus shown in FIG. 2, in accordance with an embodiment.

FIG. 4 is a front elevation view of the injector of FIG. 3, shown without the associated heating member and cap cover.

FIG. 5 is a perspective view of the injector of FIG. 3 and shown in combination with a handle and a connecting wire.

FIGS. 6 and 6a are respectively a top plan view and a front elevation view of of an injector, according to another embodiment where the injector includes a concentrated source material receptacle.

FIG. 7 is a perspective view of the gasification apparatus, according to an embodiment and shown in combination with an inhalation component.

FIG. 8 is a perspective view of the apparatus of FIG. 7, shown in combination with an alternative inhalation component.

FIG. 9 is a schematic flow chart illustrating the steps of a method for gasifying and concentrating a gasifiable compound, according to an embodiment.

### DETAILED DESCRIPTION

In the following description, the same numerical references refer to similar elements. The embodiments, geometrical configurations, materials mentioned and/or dimensions shown in the figures or described in the present description are embodiments only, given solely for exemplification purposes.

Moreover, although the embodiments of the apparatus for gasifying and concentrating a gasifiable compound and corresponding parts thereof consist of certain geometrical configurations as explained and illustrated herein, not all of these components and geometries are essential and thus should not be taken in their restrictive sense. It is to be understood, as also apparent to a person skilled in the art, that other suitable components and cooperation thereinbetween, as well as other suitable geometrical configurations, may be used for the apparatus for gasifying and concentrating a gasifiable compound, as will be briefly explained herein and as can be easily inferred herefrom by a person skilled in the art. Moreover, it will be appreciated that positional descriptions such as “above”, “below”, “left”, “right” and the like should, unless otherwise indicated, be taken in the context of the figures and should not be considered limiting.

In the course of the present description, the term “gasification” and any variation thereof should be understood to refer to the transition of a substance to the gas phase, independently from the previous phase of the substance, for instance either a liquid phase or a solid phase. Therefore, the term may be used to refer to the process of vaporization or sublimation interchangeably.

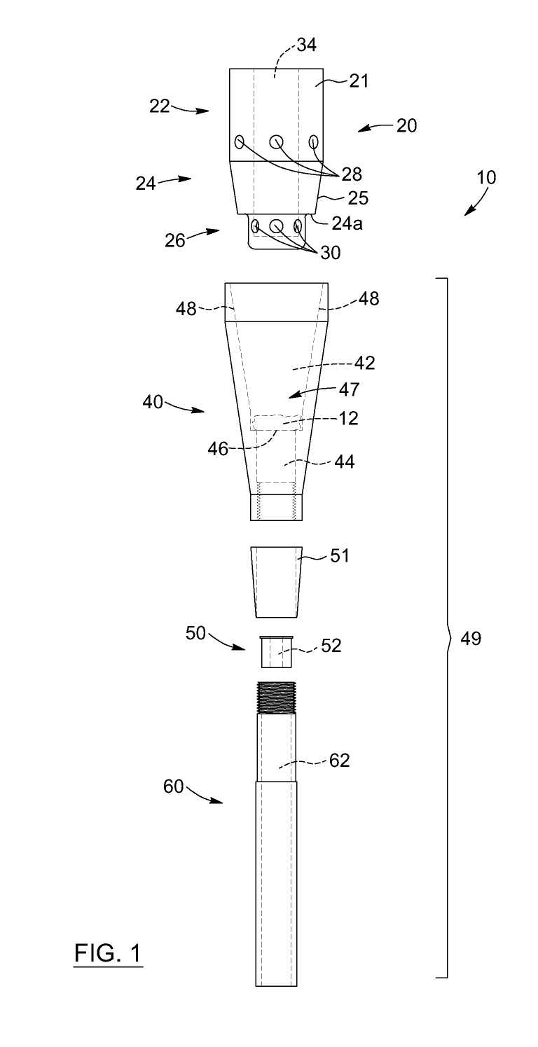

Referring generally to FIG. 1, in accordance with one embodiment, there is provided a gasification apparatus 10 for gasifying and concentrating a gasifiable compound found in a source material 12, such as, without being limitative a plant material. In the illustrated embodiment, the apparatus 10 includes an injector 20, a receiving member 40, and a concentration member 50 engageable together. The concentration member 50 comprises at least one concentration channel 52 extending therethrough. When engaged together, the injector 20, the receiving member 40, and the concentration member 50 are in gas communication with one another, in order to allow a gas flow between the components, as will be described in more details below.

One skilled in the art will understand that, even though in the course of the present description the term “injector” is used to refer to the closure member of the gasification apparatus 10, in alternative embodiments (not shown), other types of closure members engageable with the receiving member 40 could be provided.

Now referring to FIGS. 1 to 5, in an embodiment the injector 20 has an injector body 21 with an inlet (upper) section 22, an outlet (lower) section 26, and a middle section 24 extending between the inlet section 22 and the outlet section. Each one of the sections 22, 24, 26 is sized and shaped such that the injector 20 can be coupled to the receiving member 40, in a thermally conductive and gas communication assembly, as will be described below. For example and without being limitative, in the illustrated embodiment, the injector body 21 presents a substantially cylindrical configuration where the inlet section 22 has a constant diameter along its length, the middle section 24 is tapered, i.e. the middle section 24 has a decreasing diameter between the inlet section 22 and the outlet section 26 provided by an inclined outer sidewall 25, and the outlet section 26 has a constant diameter smaller than the smallest diameter of the middle section 24. Thus, an abutting shoulder 24a extends between a lower end of the middle section 24 and an upper end of the outlet section 26, the abutting shoulder 24a extending outwardly and circumscribing the upper end of the outlet section 26.

In order to allow an air flow through the injector 20, the injector body 21 also includes at least one air inlet port 28 in gas communication with at least one air outlet port 30. In the illustrated embodiment, the injector body 21 comprises a plurality of spaced-apart apertures formed in the periphery of the inlet section 22 of the injector body 21 and defining gas inlet ports 28 and a plurality of spaced-apart apertures formed in the periphery of the outlet section 26 of the injector body 21 and defining gas outlet ports 30. Such a configuration of the air inlet ports 28 and air outlet ports 30 results in the airflow moving circularly into the elements of the apparatus 10, in a vortex like movement.

The air inlet ports 28 and air outlet ports 30 are connected by through holes (not shown) extending into the injector body 21. Thus, one through hole extends between each pair of air inlet ports 28 and air outlet ports 30. In an embodiment, the quantity and the size of the through holes extending between the air inlet ports 28 and the air outlet ports 30 are configured such that the air drawn into the through holes and outputted from the air outlet ports 30 is sufficiently heated to cause the gasification of the gasifiable compound of the source material 12 by convection heating, as will be described later.

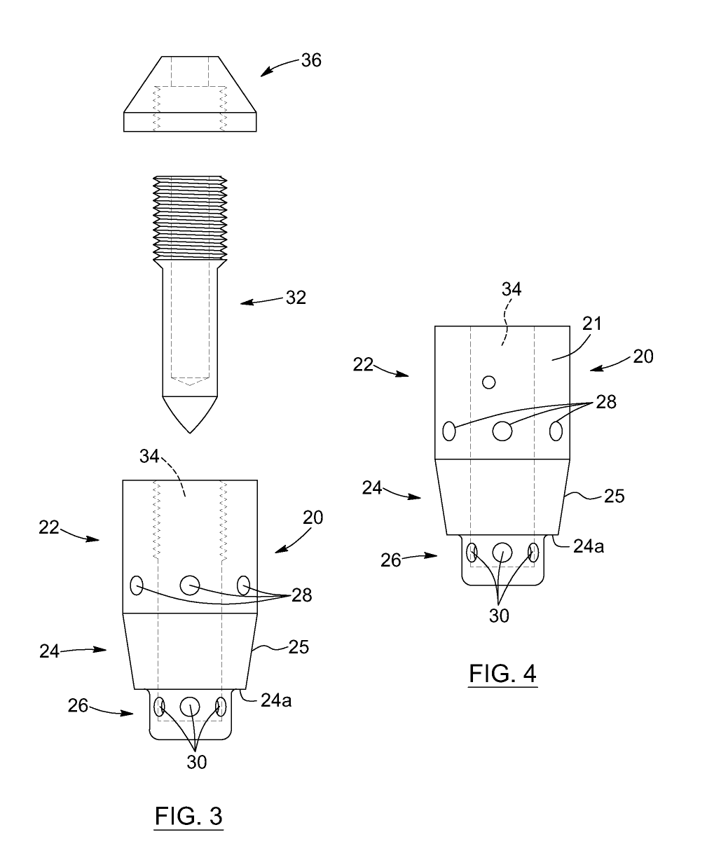

As can be better seen in FIG. 3, in an embodiment, the injector 20 is connectable to a heating member 32 which heats the injector 20. The heating of the injector 20 by the heating member 32 occurs by heat conduction between the heating member 32 and the injector body 21. In the illustrated embodiment, the heating member 32 is inserted in a central cavity 34 of the injector body 21, and is maintained in place by a cap cover 36, for example and without being limitative through a threaded connection in the cap cover, at an upper section of the heating member 32 and into the central cavity 34. One skilled in the art will easily understand that, in alternative embodiments, other arrangements for connecting the heating member 32 to the injector 20 such that the injector 20 and the heating member 32 are thermally coupled to allow the heat generated by the heating member 32 to be transferred to the injector body 21, can also be provided.

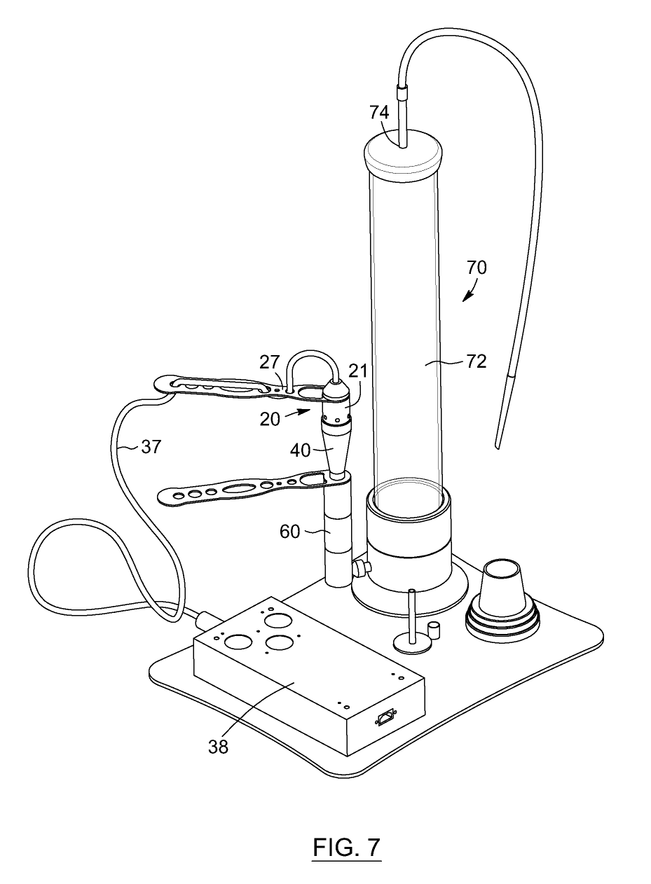

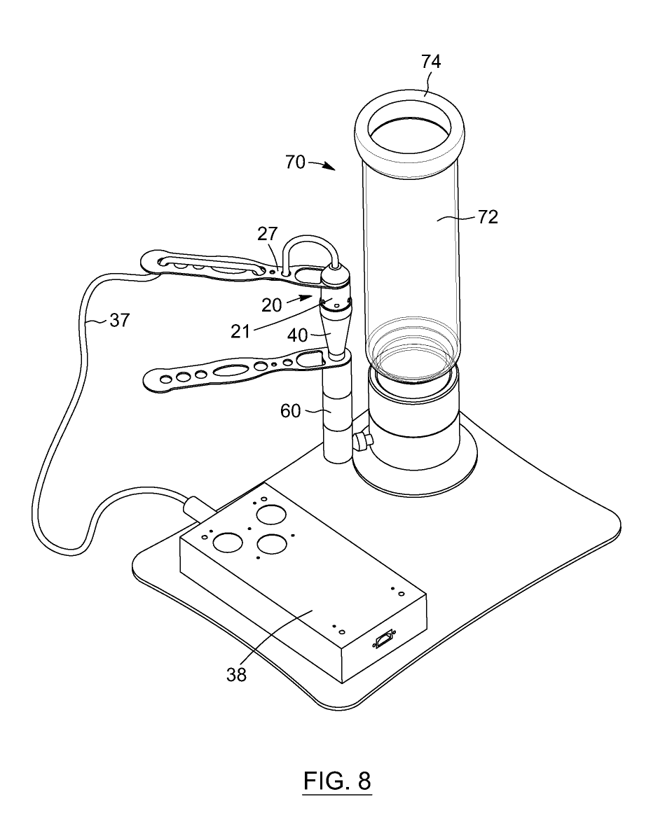

Referring to FIGS. 7 and 8, in an embodiment, the heating member 32 is an electric heating member, for a better control of the heat generated by the heating member 32, and a resulting overall better control of the temperature of the gasification apparatus 10. In such an embodiment, the heating member 32 is connected to a controller 38 by a connecting wire 37. The controller 38 controls the temperature of the heating member 32, such that the heating member 32 is brought to a temperature corresponding to a temperature set-point and is subsequently maintained at the temperature set-point. The temperature set-point is determined by the gasification temperature of the gasifiable compound comprised in the source material and which the user wishes to extract. In an embodiment, the controller 38 controls the temperature of the heating member 32 by pulsated heat control.

One skilled in the art will understand that in alternative embodiments, the heating member 32 may be any type of element allowing the injector 20 and any other additional component to which the injector 20 is connected to be heated. For example, in an embodiment (not shown), the heating member 32 may be a heat conductive protrusion extending from the injector 20 and which allows the injector 20 to be heated by heat conduction when a section of the protrusion is heated with a heating source, such as a soldering torch, a fire source, or the like.

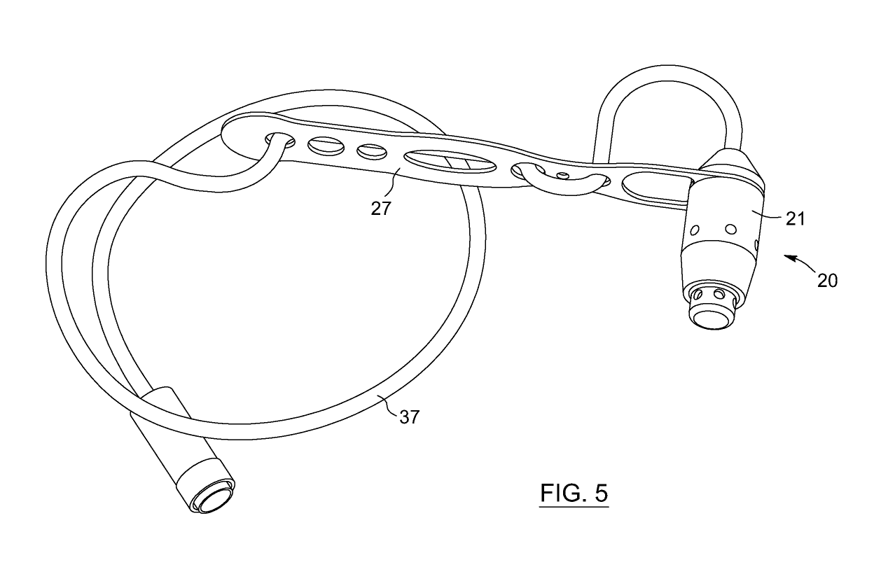

Referring to FIGS. 5, 7 and 8, in an embodiment, the injector 20 is provided with a handle 27 connected to the injector body 21. The handle 27 is made of, or is covered with, a material having a low thermal conductivity in order to allow users to manipulate the injector 20 even when the injector 20 is in a heated condition.

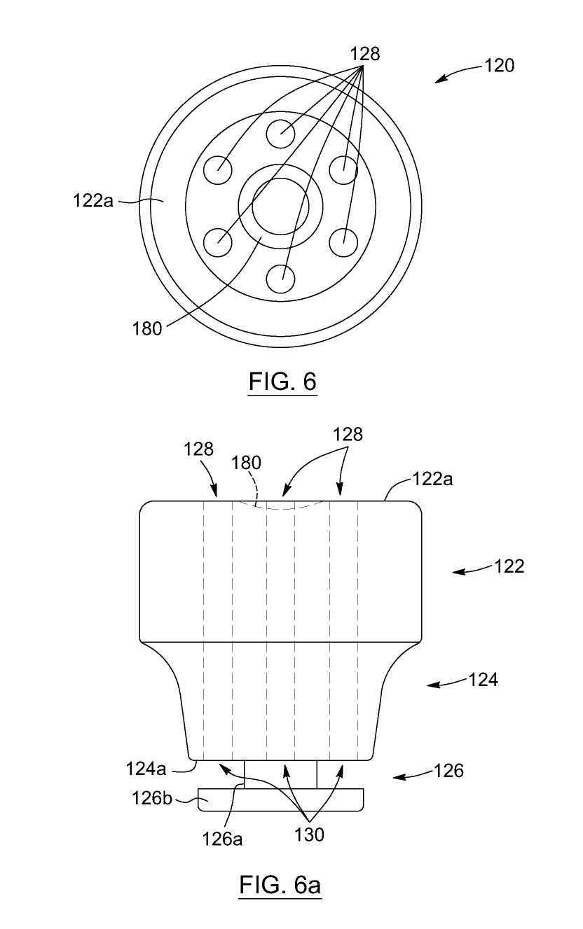

Referring to FIGS. 6 and 6a, there is shown an alternative embodiment of the injector 20 wherein the features are numbered with reference numerals in the 100 series which correspond to the reference numerals of the previous embodiment. In an embodiment shown in FIGS. 6 and 6a, the injector 120 includes a concentrated substance receptacle 180. The concentrated substance receptacle 180 is configured to allow a user to place a concentrated source material, such as, without being limitative, cannabis oil, therein. Moreover, the concentrated substance receptacle 180 is positioned relative to the air inlet ports 128 of the injector 120 such that the gas produced from the gasification of the gasifiable compound of the concentrated source material, as a result of the heating of the injector 120, flows through at least one of the air inlet ports 128 and into the gasification apparatus 10 as a result of the air ingress caused by the inhalation action of the user.

For example, in the embodiment shown in FIGS. 6 and 6a, the concentrated substance receptacle 180 is a depression defined in an inlet (upper) wall 122a of the injector 120 and is central to a plurality of air inlets 128 positioned around the concentrated substance receptacle 180. In an alternative embodiment (not shown), where the inlet ports 128 comprise apertures formed in the sidewall of the inlet (upper) section 122 of the injector body 121, the concentrated substance receptacle may comprise a slot defined in the sidewall of the injector body 121, with one of the inlet ports 28 being located inside the slot, close to a bottom of the slot. In such an embodiment, the concentrated source material can be placed inside the slot, close to the bottom thereof, for gasification.

Referring to FIG. 6a, in an embodiment, the injector 120 may also be configured such that the outlet ports 30 are formed in an abutting shoulder (lower wall) 124a of the middle section 124. In the illustrated embodiment, the outlet section 126 of the injector 120 also includes a first annular member 126a and a second annular member 126b. The first annular member 126a is connected to the abutting shoulder 124a of the middle section 124, and is sized and shaped such that the outlet ports 130 are positioned around the first annular member 126a. The second annular member 126b is connected to the first annular member 126a, at a distance from the abutting shoulder 124a of the middle section 124. The second annular member 126b also has a diameter that is greater than the diameter of the first annular member 126a, such as to extend horizontally past the outlet ports 130 formed in the abutting shoulder 24a. Such a configuration of the outlet ports 130, the first annular member 126a and the second annular member 126b also allows the above-described vortex like movement of the airflow in the elements of the gasification apparatus 10.

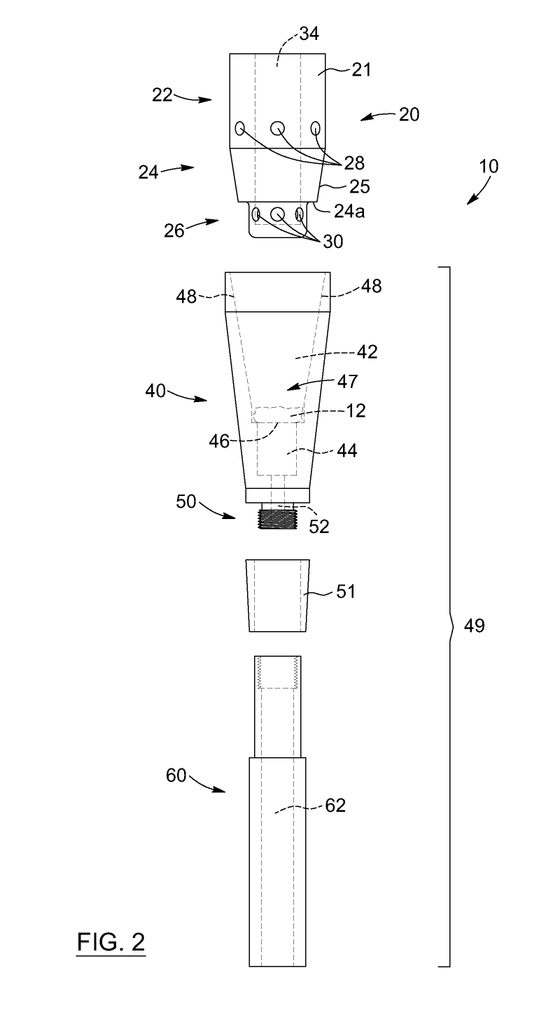

It is appreciated that either one of the injector 20, the injector 120, or any variation thereof can be used in combination with the other components of the gasification apparatus shown in FIGS. 1 and 2.

Referring back to FIGS. 1 and 2, the receiving member 40 includes a receiving section 42 and a treatment gas outlet 44 in gas communication with the receiving section 42. In the illustrated embodiment, the receiving section 42 is a cavity formed in the receiving member 40 for receiving the source material 12 therein. A gasifiable source material support 46 is provided at a bottom of the cavity, i.e. at a junction of the receiving section 42 and the treatment gas outlet 44, for depositing the source material 12 thereon. For example, the gasifiable source material support 46 can be a metal screen. The cavity is sized and shaped for receiving a portion of the injector 20 in such a way that the injector 20 closes the cavity and that the outlet section 26 of the injector 20 is close to the source material 12, without contacting it. The closed space formed therein, and where the source material 12 is inserted, may be referred to as the gasification chamber 47.

The shape of the cavity is dictated by the configuration of the sidewalls 48 of the cavity. In the illustrated embodiments, the sidewalls 48 are inclined. The inclination of the sidewalls 48 of the cavity is configured to substantially correspond to the inclination of the sidewall 25 of the middle section 24 of the injector body 21, thereby resulting in the injector 20 being maintained into the cavity in a friction fit, when inserted therein. The above described engagement of the middle section 24 and the outlet section 26 of the injector body 21 in the cavity forming the receiving section 42 of the receiving member 40 results in a portion of the cavity being unfilled to define the gasification chamber 47. This engagement of the heated injector 20 in the cavity forming the receiving section 42 of the receiving member 40 also results in the receiving member 40 being thermally coupled to the injector 20, amongst other by heat conduction, when the latter is connected to the receiving member 40.

As can be understood, in the above described configuration, the inlet ports 28 and outlet ports 30 of the injector 20 are not obstructed by the receiving member 40, when the injector 20 is inserted into the cavity defining the receiving section 42. Therefore air can be brought into the gasification chamber 47 from the air outlets 30 defined in the injector body 21.

As will be described later, the combination of the heated injector 20 with the receiving member 40 generates treatment gas from the source material 12, when source material 12 is placed into the gasification chamber 47 formed between the injector 20 and the receiving section 42. The treatment gas flows through the gasifiable source material support 46 into the treatment gas outlet 44 which is in gas communication with the receiving section 42.

It is appreciated that the shape of the injectors 20, 120 can vary from the embodiments described above in reference to the accompanying drawings. For instance, the shape of the injector body 21, 121 and the shape and the configuration of the inlet and outlet ports 28, 30, 128, 130 can vary from the embodiments shown.

One skilled in the art will understand that, in alternative embodiments, the receiving member 40 and the injector 20 may present a different configuration than the configuration illustrated in the figures and described above. For example and without being limitative, in an alternative embodiment (not shown), the receiving member 40 could present a flat upper gasifiable source material support on top of a tube and onto which the source material 12 may be deposited. The flat upper gasifiable source material support would form the receiving section 42. In this alternative configuration, the injector 20 may include a cavity formed in the outlet section thereof and shaped and sized to engage the tube onto which the flat upper gasifiable source material support rests. For example, flanges may be provided on the periphery of the tube for supporting the bottom of the injector 20. When the injector 20 is connected with the receiving member 40, the cavity formed in the outlet section of the injector 20 and the flat upper gasifiable source material support would form the gasification chamber 47 encapsulating the source material 12. In such a configuration, it is understood that the outlet ports 30 of the injector 20 would need to be provided inside the cavity formed in the outlet section of the injector 20.

Moreover, in another alternative embodiment (not shown), the injector 20 may be replaced by another type of closure member having a closure member body engageable with the receiving member 40 to form a closed gasification chamber 47. For example and without being limitative, the closure member can be a cover for covering a cavity formed in the receiving member to form the gasification chamber 47. Air intake into the gasification chamber 47 may be provided by intakes either going through the closure member or the receiving section. The heating member also can be connected either to the receiving member comprising the receiving section or to the closure member.

Still referring to FIGS. 1 and 2, a concentration member 50 in gas communication with the treatment gas outlet 44 of the receiving member 40 is further provided. In an embodiment, the concentration member 50 is also in thermal exchange (thermal communication), amongst other by heat conduction, with the receiving member 40 such that the heat transmitted to the receiving member 40 from the injector 20 is further transmitted to the concentration member 50. In an embodiment, the concentration member 50 may be connectable to the treatment gas outlet 44 of the receiving member 40 (see FIG. 1). In an alternative embodiment, the concentration member 50 may be integral with the receiving member 40 (see FIG. 2).

Therefore, once the injector 20 has reached the above-mentioned temperature set-point and has been connected to the receiving member 40 for at least an appropriate time period, the injector 20, the receiving member 40 and the concentration member 50 reach a state of substantial thermal equilibrium where all the components are approximately at the same temperature. It will be understood that the receiving member 40 and the concentration member 50 may be part of the same assembly. This assembly may be referred to as the gasification body 49.

The concentration member 50 is configured to concentrate the treatment gas generated from the source material 12 into a concentrated treatment gas, as the treatment gas flows therethrough. More particularly, in an embodiment, concentration of the treatment gas occurs as the treatment gas travels through the at least one concentration channel 52 defined in the concentration member 50. Indeed, since the at least one concentration channel 52 of the concentration member 50 has a small inner diameter, and consequently a small internal volume, the passage of the treatment gas through the at least one concentration channel 52 causes a concentration of the gasified compound contained in the treatment gas and therefore results in a concentrated treatment gas being outputted from the concentration member 50. The concentrated treatment gas has a greater concentration of gasified compound or active substance in parts-per-million than the treatment gas.

In an embodiment, the process of concentration of the gasified compound contained in the treatment gas, causes atomization of the gasified compound.

In a first embodiment, the internal volume of the at least one concentration channel 52 is between about 20 mm³ and about 50 mm³. In a second embodiment, the internal volume of the concentration channel is between about 20 mm³ and about 30 mm³. For example and without being limitative, in an embodiment, a concentration channel of a length of about 6 mm and a diameter of about 3 mm may be provided.

In an embodiment, the concentration member 50 is further connected to a concentrated treatment gas output tube 60. The concentrated treatment gas output tube 60 is in gas communication with the concentration member 50. In an embodiment, the concentrated treatment gas output tube 60 is also in thermal exchange (thermal communication) with the concentration member 50. The concentrated treatment gas output tube 60 has a concentrated treatment gas output channel 62 formed therein. The concentrated treatment gas output channel 62 has an inner diameter greater than the at least one concentration channel 52 defined in the concentration member 50 and allows the flow of concentrated treatment gas towards an inhalation component 70. In the illustrated embodiment, an optional outer ring 51 is provided. The outer ring 51 is sized and shaped to be engageable with the concentrated treatment gas output tube 60, such as to cover a section thereof, when engaged therewith. The outer ring 51 is maintained between the receiving member 40 and a lower portion of the concentrated treatment gas output tube 60 when the concentrated treatment gas output tube 60 is engaged with the receiving member 40.

In an embodiment, deposition of a portion of the gasified compound may occur in the concentrated treatment gas output tube 60, a deposited compound being formed on the periphery of the concentrated treatment gas output channel 62. In an embodiment, the concentrated treatment gas output tube 60 may be configured to allow easy collection of the deposited compound. For example, in an embodiment (not shown), the concentrated treatment gas output tube 60 may include a compound collection member. Moreover, the concentrated treatment gas output tube 60 may be combined with several subsequent sections or arrangements of pipes or tubes. The different sections may have a different inner diameter and may connect at different angles to allow easy connection with an inhalation component 70.

Referring to FIGS. 7 and 8, the inhalation component 70 is engageable with the concentrated treatment gas output tube 60 and in gas communication therewith. The inhalation component 70 has a gas channel 72 defined therein with a gas outlet port 74. When the inhalation component 70 is engaged with the concentrated treatment gas output tube 60, the gas channel 72 is in gas communication with the channel 62 of the concentrated treatment gas output tube 60 and allows a user to inhale the concentrated treatment gas by dragging the concentrated treatment gas from the gas outlet port 74. Different types of inhalation components 70 may be used, with or without a cooling process for cooling the concentrated treatment gas before it is inhaled by the user.

In an embodiment, the receiving member 40, the concentration member 50 and the concentrated treatment gas output tube 60 are removably connectable to one another, the components being connected to one another through threaded connections. One skilled in the art would however understand that in alternative embodiments, all of these components, or a subset of these components, could be integral, thereby forming a single element comprising different sections.

One skilled in the art will understand that for the components to be in thermal connection with one another, in an embodiment, the injector 20, the receiving member 40, and the concentration member 50 are made of a thermally conductive material. In an embodiment, the gasifiable source material support 46 of the receiving section 42 is also made of a thermally conductive material such that the source material 12 deposited onto it is heated by heat conduction. In an embodiment, the components are made of one of chirurgical stainless steel or titanium. In alternative embodiments, other thermally conductive and non-reactive metal or alloy may be used.

In an embodiment, the gasification apparatus 10 uses the combination of thermal conduction, convection and thermal radiation in order to perform the gasification of the gasifiable compound of the source material 12. Indeed, in operation, it is the effect of the heat generated from the heating member 32 along with the transmission of the heat to the injector 20 and the receiving member 40, and its combination with the hot air drawn into the receiving section 42 from the injector 20 which causes the initial gasification of the gasifiable compound from the source material 12. The conduction of the heat from the heating member 32 to the gasifiable source material support 46 of the receiving section 42 of the receiving member 40 causes gasification of the gasifiable compound of the source material 12 by thermal conduction. The heating of the outside air, as it flows between the air inlet ports 28 and the air outlet ports 30 of the injector body 21, produces hot air which exits the air outlet ports 30 and runs through the source material to cause gasification of the gasifiable compound of the source material 12 by convection heating. Finally, the proximity of the heated injector 20, being made of metal, with the source material 12 placed in the receiving section 42 causes gasification of the gasifiable compound of the source material 12 by radiation heating.

In an alternative embodiment, the components of the above described apparatus may be sold as an assembly kit, where a buyer assembles the components together to form the above described apparatus 10. The assembly kit may include all of the above described elements of the apparatus 10, which need not be repeated here. It should be understood that, in an embodiment, the apparatus formed by the injector 20, the corresponding heating member 32, the receiving member 40, the concentration member 50 and alternatively the concentrated treatment gas output tube 60 may be mounted to an inhalation component already owned by a user.

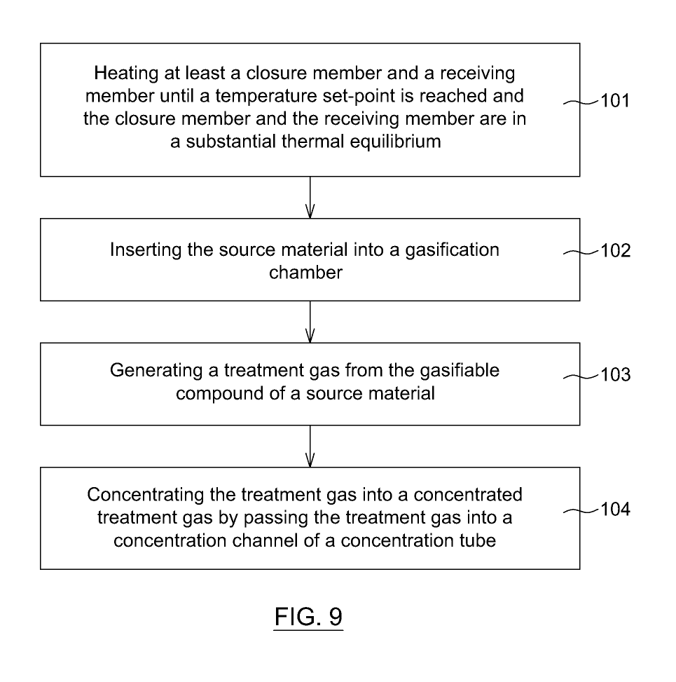

Now referring to FIG. 9, a method for gasifying and concentrating a gasifiable compound from a source material, using the above-mentioned apparatus and according to an embodiment, will be described below.

In an embodiment, in a first step 101 of the method, the closure member, the receiving member and the concentration member are heated until they have reached a temperature set-point. Once all the components have approximately reached the temperature set-point, they are said to be in a state of substantial thermal equilibrium, which is reached, amongst others, by thermal conduction between the components, when the closure member is connected to the receiving member. In an alternative embodiment, only the closure member and the receiving member may be brought in the state of substantial thermal equilibrium. Once again it will be understood that in an embodiment, the closure member is an injector.

Once again, the temperature set-point corresponds to a temperature wherein a gasifiable compound from a source material is at least partially gasified. In an embodiment where the gasifiable compound is Tetrahydrocannabinol (THC) contained in cannabis, which is the source material, the temperature set-point is between about 190° and about 240° Celsius.

In an embodiment, once the substantial thermal equilibrium is reached, the method includes the further step 102 of inserting the source material into the gasification chamber formed by the combination of the receiving section of the receiving member and the closure member. In order to insert the source material into the receiving section, the closure member may be disconnected momentarily from the receiving section to allow the insertion of the source material and be subsequently reconnected to the receiving section.

In an alternative embodiment, the source material can be inserted into the gasification chamber before the heating of the components begins, or before the components have reached the substantial thermal equilibrium.

The method further includes the step 103 of generating a treatment gas from the gasifiable compound of the source material. In such a step, hot air is drawn into the gasification chamber from air intakes of the gasification chamber, such as the ports of the injector. In an embodiment, the hot air results from the outside air being heated as it flows between the air inlet ports and the air outlet ports of the injector and is drawn from the outlet ports as a result of an induced suction effect, for example by the inhalation of a user. The combination of the hot air, the radiant heat from the closure member and the heat of the receiving section resulting from conduction causes the gasification of the gasifiable compound of the source material into a treatment gas.

In step 104, the treatment gas is subsequently drawn into the at least one concentration channel, which is characterized by a smaller diameter, of the concentration member and is concentrated into a concentrated treatment gas.

In an embodiment, the concentrated treatment gas can subsequently be delivered to the user, which inhales the concentrated treatment gas.

Several alternative embodiments and examples have been described and illustrated herein. The embodiments of the invention described above are intended to be exemplary only. A person skilled in the art would appreciate the features of the individual embodiments, and the possible combinations and variations of the components. A person skilled in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while specific embodiments have been illustrated and described, numerous modifications come to mind without significantly departing from the scope of the invention as defined in the appended claims.

1. A gasification apparatus for gasifying and concentrating a gasifiable compound, the gasification apparatus comprising:
a closure member thermally couplable to a heating member configured to heat the closure member;
a receiving member having a receiving section and a treatment gas outlet in gas communication with the receiving section, the closure member being engageable with the receiving member to close the receiving section and define a gasification chamber in between having at least one air intake, the receiving member being in thermal exchange with the closure member when they are engaged together; and
a concentration member in thermal communication with the receiving member and in gas communication with the treatment gas outlet of the receiving member, the concentration member having at least one concentration channel with an internal volume configured to cause a treatment gas flowing from the gasification chamber to concentrate into a concentrated treatment gas.

2. (canceled)

3. (canceled)

4. The gasification apparatus as claimed in claim 1, wherein the closure member comprises an injector having an injector body, the at least one air intake comprising at least one air inlet port defined in the injector body and being in gas communication with at least one air outlet port defined in the injector body, the at least one air outlet port being located in the receiving section when the closure member and the receiving member are engaged together.

5. (canceled)

6. (canceled)

7. The gasification apparatus as claimed claim 1, further comprising a concentrated treatment gas output tube connectable to the concentration member and being in gas communication with the concentration member when connected thereto, the concentrated treatment gas output tube defining a concentrated treatment gas output channel having an inner diameter greater than an inner diameter of the at least one concentration channel of the concentration member.

8. (canceled)

9. The gasification apparatus as claimed in claim 1, wherein the internal volume of the at least one concentration channel formed in the concentration member is between about 20 mm³ and about 50 mm³.

10. (canceled)

11. The gasification apparatus as claimed in claim 4, wherein the injector further comprises a concentrated source material receptacle for receiving a concentrated source material, the concentrated source material receptacle comprising a cavity defined in an inlet wall of the injector and being configured to allow at least a portion of a gas generated from the concentrated source material to be drawn into at least one of the at least one air inlet port.

12. (canceled)

13. A gasification apparatus for gasifying and concentrating a gasifiable compound, the gasification apparatus comprising:
a closure member having a closure member body;
a gasification body engageable with the closure member to define a gasification chamber in between, the gasification body being in heat conductive transfer with the closure member when engaged together, the gasification chamber being in gas communication with at least one air intake defined in at least one of the gasification body and the closure member body, the gasification body including at least one concentration channel in gas communication with the gasification chamber; and
a heating member operatively connectable to at least one of the closure member body and the gasification body for heating the at least one of the closure member body and the gasification body.

14. The gasification apparatus as claimed in claim 13, wherein the gasification body further comprises a gas permeable gasifiable source material support mounted in the gasification chamber upstream of the at least one concentration channel.

15. (canceled)

16. The gasification apparatus as claimed in claim 13, wherein the closure member comprises an injector having an injector body, the at least one air intake comprising at least one air inlet port defined in the injector body and being in gas communication with at least one air outlet port defined in the injector body, the at least one air outlet port being in gas communication with the gasification chamber when the closure member is engaged with the gasification body.

17. The gasification apparatus as claimed in claim 16, wherein the injector further comprises a concentrated source material receptacle for receiving a concentrated source material, the concentrated source material receptacle being configured to allow at least a portion of a gas generated from the concentrated source material to be drawn into at least one of the at least one air inlet port.

18. (canceled)

19. The gasification apparatus as claimed in claim 16, wherein the at least one air inlet port comprises at least one aperture defined in a periphery of an inlet section of the injector body and the at least one air outlet port comprises at least one aperture in a periphery of an outlet section of the injector body.

20. (canceled)

21. The gasification apparatus as claimed in claim 13, wherein an internal volume of the at least one concentration channel is between about 20 mm³ and about 50 mm³.

22. (canceled)

23. The gasification apparatus as claimed in claim 13, wherein the gasification body further comprises a concentrated treatment gas output tube connectable to the concentration channel and being in gas communication therewith when connected thereto, the concentrated treatment gas output tube defining a concentrated treatment gas output channel having an inner diameter greater than an inner diameter of the at least one concentration channel.

24. (canceled)

25. A gasification assembly kit for an apparatus to gasify and concentrate a gasifiable compound, the gasification assembly kit comprising:
a closure member;
a receiving member with a receiving section and a treatment gas outlet, the receiving section being sized and shaped to be connectable with the closure member to define a gasification chamber in gas communication with the treatment gas outlet, the gasification chamber being in gas communication with at least one air intake defined in at least one of the receiving section and the closure member;
a heating member thermally connectable to at least one of the closure member and the receiving member; and
a gas concentration member engageable with the receiving member and having at least one concentration channel, the at least one concentration channel being in gas communication with the treatment gas outlet when the gas concentration member and the receiving member are engaged together.

26. The gasification assembly kit as claimed in claim 25, wherein the closure member comprises an injector having an injector body, the at least one air intake comprising at least one air inlet port defined in the injector body and being in gas communication with at least one air outlet port defined in the injector body, the at least one air outlet port being in gas communication with the gasification chamber when the closure member is engaged with the receiving member.

27. The gasification assembly kit as claimed in claim 26, wherein the injector further comprises a concentrated source material receptacle for receiving a concentrated source material, the concentrated source material receptacle being configured to allow at least a portion of a gas generated from the concentrated source material to be drawn into at least one of the at least one air inlet port.

28. (canceled)

29. (canceled)

30. (canceled)

31. The gasification assembly kit as claimed in claim 25, wherein the receiving member further comprises a gas permeable gasifiable source material support mounted in the receiving section between the at least one air intake and the treatment gas outlet.

32. (canceled)

33. The gasification assembly kit as claimed in claim 25, wherein an internal volume of the concentration channel is between about 20 mm³ and about 50 mm³.

34. (canceled)

35. The gasification assembly kit as claimed in claim 25, further comprising a concentrated treatment gas output tube connectable with the gas concentration member and being in gas communication the gas concentration member when connected together, the concentrated treatment gas output tube defining a concentrated treatment gas output channel having an inner diameter greater than an inner diameter of the at least one concentration channel of the gas concentration member.

36. The gasification assembly kit as claimed in claim 35, further comprising an inhalation component connectable to the concentrated treatment gas output tube and being in gas communication therewith when connected together, the inhalation component having a gas channel defined therein with a gas outlet port.

37. A method of gasifying and concentrating a gasifiable compound found in a source material, the method comprising the steps of:
heating at least a closure member and a receiving member until a temperature set-point is reached and the closure member and the receiving member are in a substantial thermal equilibrium;
inserting the source material into a gasification chamber defined between the closure member and the receiving member, when engaged together;
generating a treatment gas from the gasifiable compound of the source material with the closure member and the receiving member being engaged together; and
concentrating the treatment gas into a concentrated treatment gas by passing the treatment gas into at least one concentration channel of a concentration member in gas communication with the gasification chamber.

38. (canceled)

39. (canceled)

40. The method as claimed in claim 37, wherein the temperature set-point corresponds to a temperature wherein the gasifiable compound at least partially gasifies.

41. The method as claimed in a claim 40, wherein the temperature set-point is between about 190° Celsius and about 240° Celsius.

42. The method as claimed in claim 37, wherein the step of heating at least the closure member and the receiving member until a temperature set-point is reached further comprises heating the concentration member until the closure member, the receiving member and the concentration member are in a substantial thermal equilibrium.

43. The method as claimed in claim 37, wherein the step of generating a treatment gas is performed by at least one of conduction heating, convection heating and radiation heating between the closure member, the receiving member, and the source material.

44. The method as claimed in claim 43, wherein the step of generating a treatment gas is performed by at least two of conduction heating, convection heating and radiation heating between the closure member, the receiving member, and the source material.

45. The method as claimed in claim 44, wherein the step of generating a treatment gas is performed by conduction heating, convection heating and radiation heating between the closure member, the receiving member, and the source material.

46. (canceled)
